(19) United States
(12) Patent Application Publication
(10) Pub. No.: US 2005/0164983 A1
(43) Pub. Date: Jul. 28, 2005

(54) Carboxyl-reduced derivatives of dermatan sulfate, preparation thereof, use thereof as a medicinal product and the pharmaceutical compositions containing them

(75) Inventors: Wolfgang Ulmer, Eppstein (DE); Christian Viskov, Ris Orangis (FR); Philippe Hubert, Maisons-Alfort (FR)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma S.A., Antony (FR)

(21) Appl. No.: 11/016,501

(22) Filed: Dec. 17, 2004

Related U.S. Application Data
(60) Provisional application No. 60/618,667, filed on Oct. 12, 2004.

(30) Foreign Application Priority Data
Dec. 19, 2003 (FR) 0314986

(51) Int. Cl. A61K 31/737

(52) U.S. Cl. 514/54; 536/54

(57) ABSTRACT

The present invention relates to the carboxyl-reduced derivatives of dermatan sulfate of formula (I): 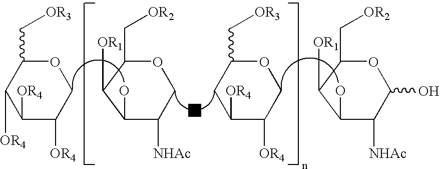
in which R₁, R₂, R₃ or R₄ represents H or SO₃M, n is an integer of between 0 and 150, M is an alkali metal, isolated or as mixtures, to the diastereoisomers thereof, to the process for the preparation thereof, to the uses thereof as a medicinal product and to the pharmaceutical compositions containing them.

This application claims the benefit of U.S. Provisional Application No. 60/618,667 filed Oct. 12, 2004 and benefit of priority from French Application 03 14986, filed Dec. 19, 2003 both of which are incorporated herein by reference in their entirety.

The present invention relates to novel glycosaminoglycans and also to their pharmaceutically acceptable addition salts, and more precisely to carboxyl-reduced and chemoselectively O-sulfated derivatives (compounds) of dermatan sulfate, isolated or as a mixture, to the use thereof as a medicinal product and to the pharmaceutical compositions containing them. Glycoaminoglycans (GAGs) are essentially made up of alternating uronic acid-amino sugar (or vice versa) units of the type such as those encountered in the oligosaccharide or polysaccharide chains of biologically active natural GAGs such as heparin, heparan sulfate, dermatan sulfate, chondroitins, chondroitin sulfates or hyaluronic acid.

The uronic acid units correspond more specially to the D-glucoronic or L-iduronic acid structure and the amino sugar units to the D-galactosamine structure.

The natural GAGs exhibit therapeutic activities as thrombin inhibitors. They therefore exhibit antithrombotic and anticoagulant activity and are used in cardiovascular pathologies in which there is a risk of thrombosis.

O-persulfated derivatives of dermatan sulfate have been described and studied for their anticoagulant properties in French patent application FR 2584728.

Moreover, O-persulfated derivatives of dermatan sulfate have been described and studied for their properties in rheumatoid arthritis in international application WO 92/13541-A1.

A subject of the present invention is a novel process using a carboxyl reduction step and a sulfatation (sulfating) step for obtaining novel carboxyl-reduced and chemoselectively O-sulfated derivatives of dermatan sulfate exhibiting advantageous properties for treating or preventing disorders which display an increased activity of at least one of the matrix metalloproteinases.

The carboxyl-reduced and chemoselectively O-sulfated derivatives of dermatan sulfate of formula (I) have a very regular polymeric structure with degrees of purity of the order of 90%. Reproducible and unexpected biological properties result therefrom.

In the pathological condition of osteoarthritis, the degradation of aggrecan, the main proteoglycan of cartilage in the joints, represents a very early and crucial event. The pathological loss of aggrecan is caused by proteolytic cleavages in its interglobular domain. Amino acid sequence analyses of the proteoglycan metabolites isolated from the synovial fluid of patients suffering from joint damage, from osteoarthritis or from an inflammatory disorder of the joints, have shown that proteolytic cleavage exists between the amino acids Glu³⁷³ and Ala³⁷⁴ in the interglobular domain of human aggrecan (Lohmander, et al., Arthritis Rheum., 36:1214-1222 (1993)). The proteolytic activity responsible for this cleavage is called “aggrecanase” and can be attributed to the metalloproteinase (MP) or matrix metalloproteinase (MMP) superfamily.

Zinc is an essential element in the catalytically active center of metalloproteinases. MMPs cleave collagen, laminin, proteoglycans, elastin or gelatin under physiological conditions. They therefore play an important role in bone tissue and connective tissue. A large number of different MMP inhibitors are known (see, for example, patent application EP 0 606 046 or WO 94/28889). However, the known MMP inhibitors frequently have a significant disadvantage. They lack specificity for any particular class of MMP. On the contrary, most MMP inhibitors simultaneously inhibit a plurality of MMPs.

Consequently, a need exists for MMP inhibitors that have more narrowly defined specificities in order to more effectively treat or prevent specific disorders.

A subject of the invention is most particularly the carboxyl-reduced and sulfated derivatives of dermatan sulfate of formula (I): 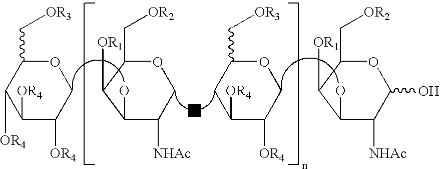
in which R₁ represents SO₃M, R₂, R₃ and R₄, which may be identical or different, represent H or SO₃M, it being understood that at least one of the substituents R₂, R₃ and R₄ represent a group SO₃M, n is an integer of between 0 and 150, M is an alkali metal, said derivatives being in the form of an isolated compound or in the form of mixtures, and also the diastereoisomers thereof.

In particular, a subject of the invention is the carboxyl-reduced derivatives of dermatan sulfate according to formula (I), wherein M is chosen from sodium, calcium, magnesium and potassium, and in particular sodium.

A subject of the invention is more particularly the carboxyl-reduced derivatives of dermatan sulfate as described above, wherein R₂ and R₃ represent SO₃Na and R₄ represents H.

A subject of the invention is more particularly the carboxyl-reduced derivatives of dermatan sulfate as described above, wherein R₂, R₃ and R₄ represent SO₃Na.

A subject of the invention is more particularly the carboxyl-reduced derivatives of dermatan sulfate as described above, wherein R₂ and R₄ represent SO₃Na and R₃ represents H.

A subject of the invention is more particularly the carboxyl-reduced derivatives of dermatan sulfate as described above, wherein R₂ represents SO₃Na and R₃ and R₄ represent H.

These polysaccharides thus comprise an even number of saccharides.

In general, the mixtures of polysaccharides according to the invention can be prepared, firstly, by carboxyl reduction of dermatan sulfate or of its derivatives, followed by chemoselective sulfatation, or conversely by sulfatation followed by carboxyl reduction.

A subject of the invention is therefore the carboxyl-reduced and sulfated derivatives of dermatan sulfate as defined above, using the following steps in succession:
trans-salification (salt exchange) of dermatan sulfate with a quaternary ammonium salt,
sulfatation in organic medium, followed by salification,
carboxyl reduction of the persulfated derivative
   either a) by means of a carbodiimide derivative (so as to form an activated adduct) and in the presence of a reducing agent,
   or b) by esterification, the persulfated derivative being, where appropriate, trans-salified beforehand with a quaternary ammonium salt, followed by a reduction of the corresponding ester derivative by the action of a reducing agent,
where appropriate, trans-salification of the persulfated, carboxyl-reduced derivative with a quaternary ammonium salt, and then resulfatation (resulfating) followed by salification (salt formation).

The following reaction scheme illustrates the present invention: 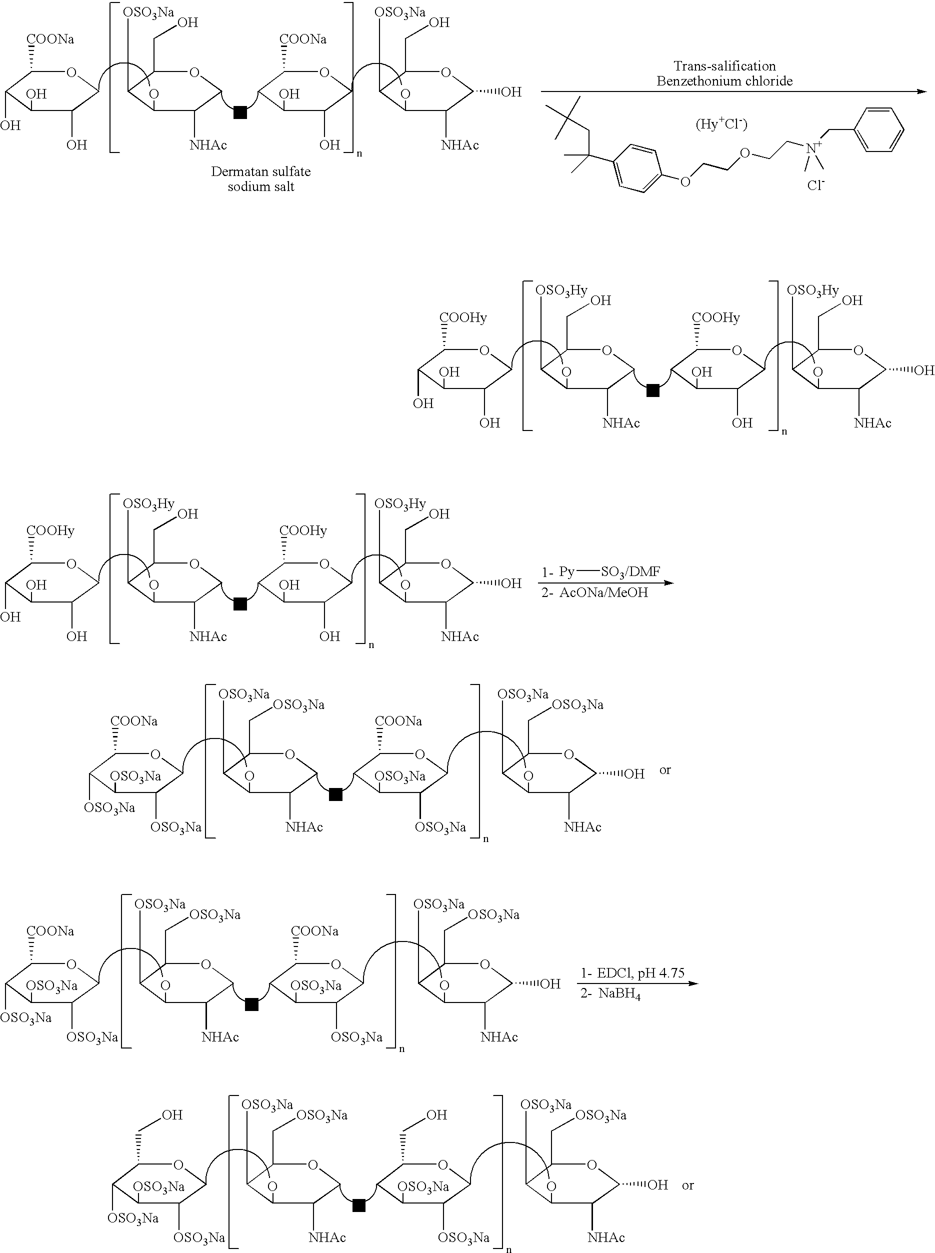 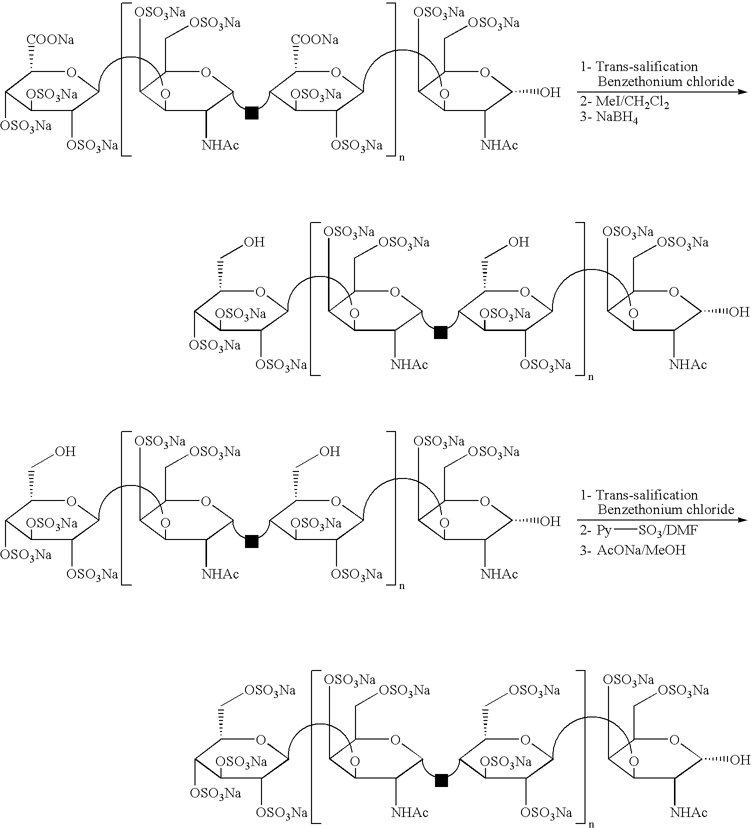
Where appropriate, the carboxyl-reduced derivatives of dermatan sulfate as defined above may be obtained according to the process carrying out the above described steps, but combined in a reverse order. The process is then as follows:
carboxyl reduction of the dermatan sulfate sodium salt,
either a) by means of a carbodiimide derivative (so as to form an activated adduct) and in the presence of a reducing agent,
   or b) by esterification, the dermatan sulfate sodium salt being, where appropriate, trans-salified beforehand with a quaternary ammonium salt, followed by reduction of the corresponding ester derivative by the action of a reducing agent,
trans-salification of the carboxyl-reduced derivative with a quaternary ammonium salt,
sulfatation in organic medium, followed by salification.

The following reaction scheme illustrates the present invention: 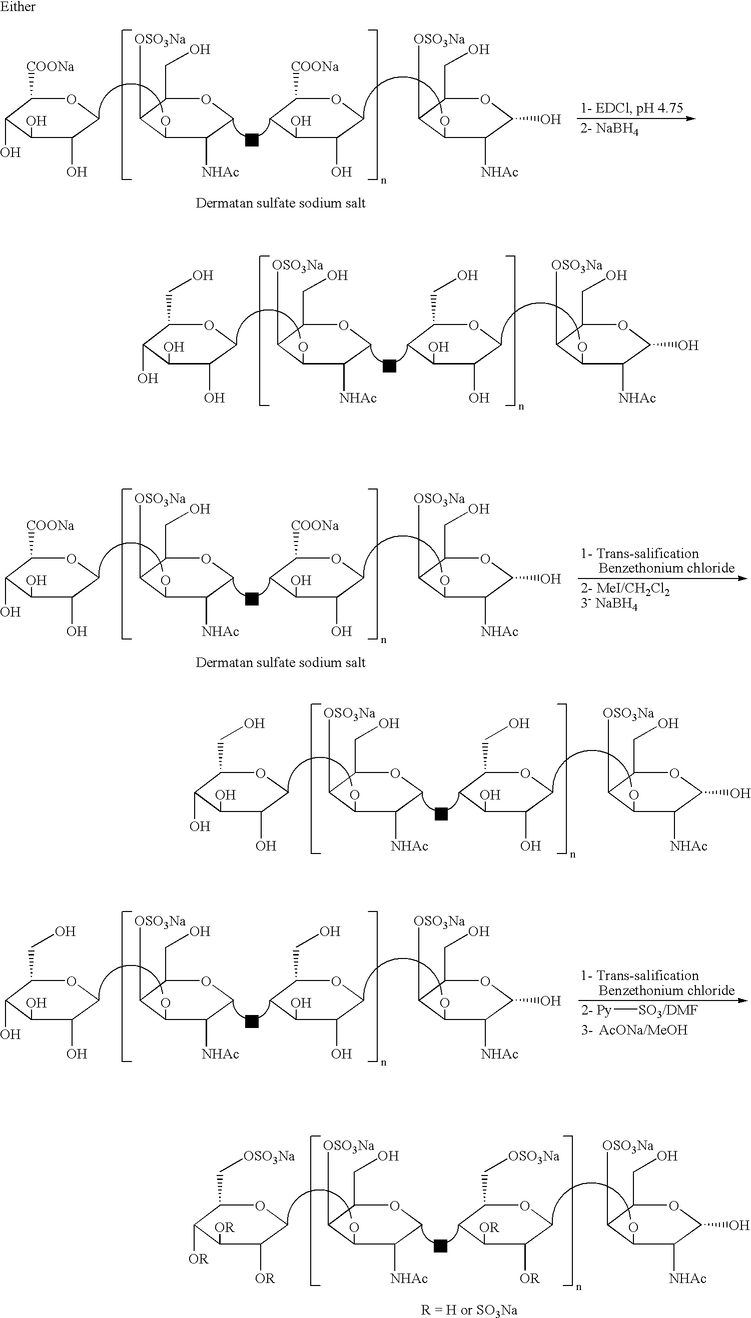
The process according to the present invention is characterized by the strong chemoselectivity of the carboxyl reduction and sulfatation reactions. The products which result therefrom are notably homogeneous and result in true polymers. The carboxyl-reduced dermatan sulfate derivatives have purities of the order of 90%. This homogeneity is determined by NMR and infrared structural analysis.

Reproducible and unexpected biological properties result therefrom.

The persulfatation reactions are preferably carried out in organic medium by means of a complex of sulfuric anhydride with an organic base such as pyridine or trimethylamine. They are generally followed by a salification reaction, for example by the action of sodium acetate.

For optimal chemoselectivity of the reactions for persulfatation, it is preferable to use the benzethonium salt of dermatan sulfate, whether carboxyl-reduced or not, in the presence of 10 to 30 equivalents of pyridine-sulfuric anhydride complex per hydroxyl function to be sulfated. Similarly, the reaction will preferably be carried out at temperatures of between −15 and 70° C.

A subject of the invention is more particularly a process for the sulfatation of dermatan sulfate, which is carried out at approximately 0° C. when it is desired to obtain the 6-O-sulfated derivatives of dermatan sulfate or to approximately 60° C. when it is desired to obtain the persulfated derivatives of dermatan sulfate. This is illustrated, respectively, in examples 4 and 5.

For optimal chemoselectivity of the 6,6′-O-sulfatation of the carboxyl-reduced dermatan sulfate benzethonium salt (compounds of formula (I) with R₂=R₃=SO₃Na and R₄=H obtained from compounds of formula (I) with R₂=R₃=R₄=H), the procedure is preferably carried out in the presence of 5 to 15 equivalents of pyridine-sulfuric anhydride complex, at temperatures of between −15 and 5° C. and in particular at around 0° C. This is illustrated in example 2.

During the persulfatation of the benzethonium salt of the carboxyl-reduced dermatan (compounds of formula (I) with R₂=R₃=R₄=SO₃Na obtained from compounds of formula (I) with R₂=R₃=R₄=H), the procedure is preferably carried out at between 50 and 70° C. This is illustrated in example 3.

Consequently, a subject of the invention is also a process for the sulfatation of the carboxyl-reduced dermatan sulfate, which is carried out at approximately 0° C. when it is desired to obtain the 6,6′-O-sulfated compounds as defined above (R₂=R₃=SO₃Na and R₄=H) or at approximately 60° C. when it is desired to obtain the persulfated compounds as defined above (R₂=R₃=R₄=SO₃Na).

During the second sulfatation of the persulfated, carboxyl-reduced derivative, benzethonium salt (compounds of formula (I) with R₂=R₄=SO₃Na and R₃=H), in order to obtain the compounds of formula (I) with (R₂=R₃=R₄=SO₃Na) the procedure is preferably carried out in the presence of 10 to 30 equivalents of pyridine-sulfuric anhydride complex, at temperatures in the region of 20° C. This is illustrated in example 6.

Consequently, a subject of the invention is also the process as defined above, wherein the sulfatation of the carboxyl-reduced, trans-salified, sulfated dermatan sulfate (compounds of formula (I) with R₂=R₄=SO₃Na and R₃=H) is carried out at approximately 20° C. when it is desired to obtain the persulfated compounds (compounds of formula (I) with R₂=R₃=R₄=SO₃Na).

Similarly, for optimal chemoselectivity of the 6′-O-sulfatation of the 6-O-sulfated, carboxyl-reduced dermatan benzethonium salt (compounds of formula (I) with R₂=SO₃Na and R₃=R₄=H), in order to obtain the compounds of formula (I) with R₂=R₃=SO₃Na and R₄=H, it is preferable to carry out the reaction in the presence of 5 to 15 equivalents of pyridine-sulfuric anhydride complex, at temperatures of between −15 and 5° C. Even more preferably, the reaction should be carried out in the presence of approximately 10 equivalents of pyridine-sulfuric anhydride complex at a temperature in the region of −10° C. This is illustrated in example 7.

A subject of the invention is therefore the process as defined above, wherein the 6′-O-sulfatation of the carboxyl-reduced, trans-salified, 6-O-sulfated dermatan sulfate (compounds of formula (I) with R₂=SO₃Na and R₃=R₄=H) is carried out at approximately 10° C. when it is desired to obtain the 6,6′-O-sulfated compounds (compounds of formula (I) with R₂=R₃=SO₃Na and R₄=H).

The carboxyl reduction of the derivatives of dermatan sulfate is carried out in the presence of a carbodiimide derivative. By way of example, use may be made of 1-ethyl-3-(3-dimethylaminopropyl)carbodiimide hydrochloride.

The term “derivatives of dermatan sulfate” is intended to mean the sulfated derivatives of dermatan sulfate, the dermatan sulfate benzethonium salt, the persulfated derivatives of dermatan sulfate benzethonium salts and also the dermatan sulfate sodium salt.

The reduction per se is carried out with an alkali metal borohydride. By way of example, the reaction with the carbodiimide derivative is carried out with sodium borohydride.

The reaction for formation of the adduct with the carbodiimide derivative is preferably carried out in the presence of 5 to 20 equivalents of reactant and at a pH of between 4 and 5.

Even more preferably, the reaction with the carbodiimide derivative is carried out in the presence of 7 to 13 equivalents of reactant and at a pH of between 4.3 and 4.9.

The reduction per se of the activated adduct of the dermatan sulfate derivatives is carried out with 10 to 300 equivalents of alkali metal borohydride at a temperature of between 10 and 70° C. More preferably, the reduction of the abovementioned adduct is carried out in the presence of 140 to 250 equivalents of alkali metal borohydride at a temperature of between 10 and 30° C.

According to another embodiment of the present invention, the carboxyl reduction can be carried out by reduction of an ester of the dermatan sulfate derivatives. By way of example, it is possible to use a methyl ester derivative.

The derivatives of dermatan sulfate presalified in the form of a benzethonium salt are esterified by conventional methods of esterification known to those skilled in the art using an alkyl halide containing from 1 to 6 carbon atoms, such as methyl iodide, or an arylalkyl halide, such as benzyl chloride, in organic medium. The esterification per se of the dermatan sulfate benzethonium salt is carried out in organic medium in the presence, for example, of methyl iodide. Preferably, the esterification is carried out in dichloromethane in the presence of 2 to 20 equivalents of methyl iodide and at a temperature in the region of 20° C.

In particular, the esterification is carried out in dichloromethane in the presence of 4 to 10 equivalents of methyl iodide for approximately 3 days at a temperature in the region of 20° C.

Similarly, the reduction of the methyl ester of the dermatan sulfate benzethonium salt derivative is carried out with 10 to 300 equivalents of alkali metal borohydride at a temperature of between 10 and 70° C. By way of example, the reduction of the methyl ester is carried out with sodium borohydride.

More preferably, the reduction of the methyl ester is carried out in the presence of 140 to 250 equivalents of alkali metal borohydride at a temperature in the region of 20° C.

In particular, a subject of the invention is the following process:
trans-salification of the dermatan sulfate with benzethonium chloride,
sulfatation of the dermatan sulfate benzethonium salt in organic medium by means of a complex of sulfuric anhydride with an organic base such as pyridine or trimethylamine, followed by salification with sodium acetate,
either carboxyl reduction by means of 1-(3-dimethylaminopropyl)-3-ethylcarbodiimide hydrochloride in the presence of sodium borohydride,
or esterification by the action of methyl iodide on the dermatan sulfate derivative trans-salified beforehand with benzethonium chloride, then reduction of the methyl ester derivative with sodium borohydride,
then, where appropriate, trans-salification of the persulfated, carboxyl-reduced derivative with benzethonium chloride, then resulfatation with the sulfuric anhydride-pyridine complex, followed by salification with sodium acetate.

Alternatively, a subject of the invention is also the process as defined above, using the following steps:
either carboxyl reduction of the dermatan sulfate sodium salt by means of 1-(3-dimethylaminopropyl)-3-ethylcarbodiimide hydrochloride in the presence of sodium borohydride,
or esterification by the action of methyl iodide on the dermatan sulfate derivative trans-salified beforehand with a quaternary ammonium salt, and then reduction of the methyl ester derivative with sodium borohydride, then
trans-salification of the carboxyl-reduced derivative with benzethonium chloride,
sulfatation with the sulfuric anhydride-pyridine complex, followed by salification with sodium acetate.

When it is desired to obtain isolated derivatives from the mixture of carboxyl-reduced derivatives of dermatan sulfate as obtained according to the process described above, the following process should then be applied to the mixture:
fractionation of the mixture by chromatography on columns filled with gel of polyacrylamide agarose type or a polyacrylamide gel. The mixture is eluted with a sodium hydrogen carbonate solution.

The sodium hydrogen carbonate solution is preferably a solution of 0.1 to 1 mol/l. Even more preferably, the separation is carried out at a concentration of 1 mol/l. The detection is carried out by refractometry.

A subject of the invention is also the carboxyl-reduced and chemoselectively 0-sulfated derivatives of dermatan sulfate, isolated or as a mixture as defined above, which can be obtained according to the process(es) as defined above.

The polysaccharides of formula (I), isolated or as mixtures, can be used as medicinal products.

These polysaccharides exhibit in particular strong inhibitory activity on certain matrix metalloproteases. These inhibitors are particularly indicated for the treatment of pathological states where a large increase in matrix metalloproteinase activity is noted.

The pathological states to which the present invention refers involve an increase in the activity of at least one of the following matrix metalloproteinases: matrisilyn (MMP-7), neutrophil elastase, aggrecanase, hADAMTS1 and gelatinase A (MMP-2).

The compounds can therefore be used for preventing and treating diseases such as degenerative joint disorders (such as osteoarthritis), spondylosis, chondrolysis associated with joint trauma or prolonged joint immobilizations (often following an injury to the meniscus or patella or the rupture of ligaments), disorders related to injuries (healing), periodontal disorders, chronic disorders of the locomotor system (such as chronic or acute inflammatory, immunological or metabolic forms of arthritis), arthropathies, myalgias or disorders related to bone metabolism.

A subject of the invention is also the pharmaceutical compositions containing the compounds of formula (I), isolated or as a mixture, and also one or more pharmaceutically acceptable excipients, vehicles or additives.

Another aspect of the invention is a process for preparing the pharmaceutical compositions containing the compounds of formula (I), wherein an amount corresponding to a desired dose of a compound of formula (I) is mixed with one or more pharmaceutically acceptable excipients, vehicles or additives.

The compounds of formula (I) can be administered via various routes. They may include, without being limited, subcutaneous, intra-articular, intraperitoneal or intravenous injections.

The administration may also be rectal, oral, by inhalation, or else transdermal.

In the case of solutions for injection (for example in the form of an ampoule), the doses may range from 5 μg to approximately 200 mg of compound of formula (I), and preferably from 10 μg to 40 mg.

The daily dose indicated for the treatment of an adult patient weighing approximately 70 kg is from 10 μg to 500 mg of active ingredients, generally from 20 mg to approximately 100 mg. However, depending on the circumstances, higher or lower daily doses may be appropriate.

These doses can be administered once a day in the form of a single dosage unit.

Alternatively, the doses can be administered in a plurality of smaller doses, given repeatedly at defined intervals over time.

The following examples illustrate the invention without, however, limiting it.

Dermatan Sulfate

The dermatan sulfate used for preparing the compounds illustrating this invention has a molecular mass of 30 to 40 K Daltons. It is isolated during the preparation of porcine mucosal heparin. A mixture of heparinoids/dermatan sulfate can be isolated from the mother liquors from precipitation of the crude heparin. To obtain pure dermatan sulfate, the following conditions may, for example, be used:

A solution of 13 g of heparinoid/dermatan mixture (containing approximately 50% of dermatan) in 130 ml of water is prepared. The pH is then adjusted to pH 3 with concentrated acetic acid and then 0.9 g of sodium nitrite is added at a temperature in the region of 20° C. After stirring for 18 hours, the reaction medium is cooled to a temperature in the region of 0° C., and then neutralized to pH 7 with concentrated sodium hydroxide (7.5N). The reaction medium is adjusted to a sodium chloride content of 10% and then one volume of methanol (i.e. approximately 130 ml) is added. The precipitate obtained is filtered off, washed with 5 ml of methanol, and then dried so as to give 4.8 g of an off-white solid. The yield obtained is 73%.

### EXAMPLE 1

### Preparation of the Carboxyl-reduced Dermatan Sulfate Sodium Salt

(Compounds of formula (I) with the groups R₂=R₃=R₄=H)

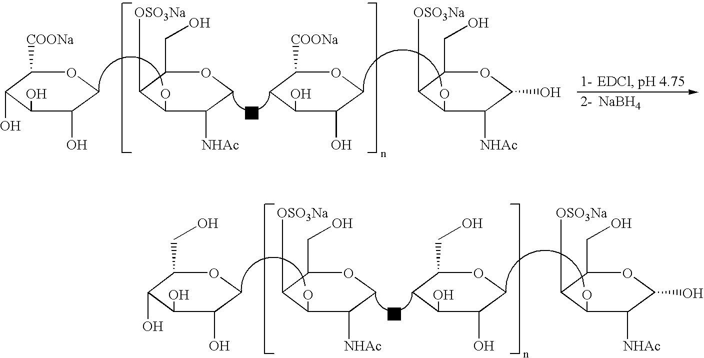

A solution of 1 g of dermatan sulfate sodium salt in 150 ml of water is prepared at a temperature in the region of 20° C. The pH is adjusted to pH 4.7±0.1 with a 0.1N hydrochloric acid solution. 3.6 g of 1-ethyl-3-(3-dimethylaminopropyl)carbodiimide hydrochloride are added, and the mixture is then stirred while maintaining the pH 4.7±0.1. When the pH no longer changes, 14.7 g of sodium borohydride are then added in small portions. The reaction medium is stirred for approximately 20 hours at a temperature in the region of 20° C. After cooling to a temperature in the region of 15° C., the reaction medium is neutralized with concentrated hydrochloric acid, at a pH in the region of 7. The reaction medium is loaded into an MW 1000 cellulose ester membrane and dialyzed, changing the water baths regularly for approximately 48 hours (the fourth bath is carried out with a 2N sodium chloride solution. The last three baths are carried out with deionized water). The content of the membranes is lyophilized. 0.8 g of a white lyophilized material is obtained. The yield obtained is 92%.

Proton spectrum in D₂O, 400 MHz, T=323 K (δ in ppm -mixture of the compounds epimerized at C5): 2.0 (3H, s), 3.5 (1H, m), 3.6 (2H, s), between 3.67 and 3.8 (5H, m), 3.94 (1H, t, J=6 Hz), 3.98 (1H, d, J=6 Hz), 4.32 (1H, s), 4.54 (1H, d, J=5 Hz), 4.7 (1H, s), 4.76 (1H, s).

### EXAMPLE 2

### Preparation of the Carboxyl-reduced, 6,6′-O-sulfated Dermatan Sulfate Sodium Salt

(Compounds of formula (I) with the groups R₂=R₃=SO₃Na and R₄=H) 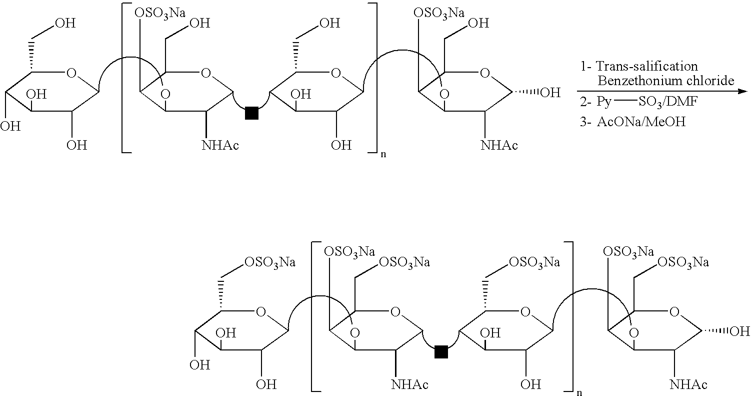
a) Preparation of the Carboxyl-reduced Dermatan Sulfate Sodium Salt

The carboxyl-reduced dermatan sulfate sodium salt is prepared according to example 1.

b) Preparation of the Carboxyl-reduced Dermatan Sulfate Benzethonium Salt

A solution of 1 g of benzethonium chloride in 7 ml of water is added to a solution of 0.4 g of carboxyl-reduced dermatan sulfate sodium salt in 6 ml of water. The white suspension is filtered. The cake is washed with 3 portions of 15 ml of water, and then dried at approximately 40° C. under reduced pressure (6 kPa). 0.73 g of a translucent yellowish solid is obtained. The yield obtained is quantitative.

c) Preparation of the Carboxyl-reduced, 6,6′-O-sulfated Dermatan Sulfate Sodium Salt

0.73 g of carboxyl-reduced dermatan sulfate benzethonium salt is dissolved in 20 ml of anhydrous dimethylformamide at a temperature in the region of 20° C. and under an inert atmosphere. The solution obtained is cooled to approximately 0° C. and then a solution of 1.23 g of pyridine-sulfuric anhydride complex in 10 ml of anhydrous dimethylformamide is run in. After stirring for 1 hour 30 min at a temperature in the region of 0° C., 15 ml of water are added to the reaction medium. 150 ml of a methanolic solution containing 10% of sodium acetate are then run in. The suspension obtained is filtered and the cake is then washed with 10 ml of methanol. The solid obtained is dissolved in 50 ml of water and then re-precipitated with 150 ml of a methanolic solution containing 10% of sodium acetate. The suspension is filtered and the cake is then washed with 10 ml of methanol. The white solid obtained is dissolved in 10 ml of water and the solution is then loaded into an MW 3500 cellulose ester membrane and dialyzed, changing the water baths regularly for 72 hours. The content of the membranes is lyophilized. 55 mg of a white lyophilized material are obtained. The yield obtained is 10%.

Proton spectrum in D₂O, 400 MHz, T=333 K, 6 in ppm: 2.0 (3H, s), 3.49 (1H, m), 3.8 (1H, m), between 3.9 and 4.25 (8H, m), 4.45 (1H, m), 4.57 (1H, broad s), 4.75 (1H, s), 4, 4.80 (1H, s).

### EXAMPLE 3

### Preparation of the Carboxyl-reduced and Persulfated Dermatan Sulfate Sodium Salt

(Compounds of formula (I) with the groups R₂=R₃=R₄=SO₃Na) 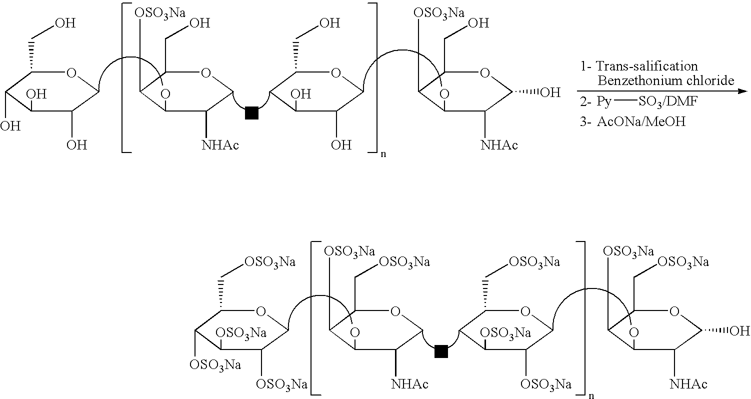
a) Preparation of the Carboxyl-reduced Dermatan Sulfate Sodium Salt

The carboxyl-reduced dermatan sulfate sodium salt is prepared according to example 1.

b) Preparation of the Carboxyl-reduced Dermatan Sulfate Benzethonium Salt

The carboxyl-reduced dermatan sulfate benzethonium salt is prepared according to example 2b.

c) Preparation of the Carboxyl-reduced and Persulfated Dermatan Sulfate Sodium Salt

0.24 g of carboxyl-reduced dermatan sulfate benzethonium salt is dissolved in 5 ml of anhydrous dimethylformamide at a temperature in the region of 60° C. and under an inert atmosphere. When the dissolution is complete, a solution of 2.94 g of pyridine-sulfuric anhydride complex in 12 ml of anhydrous dimethylformamide is run in. The mixture is stirred for 2 hours 30 min at a temperature in the region of 60° C., and then cooled to a temperature in the region of 10° C. 6 ml of water are added to the reaction medium, followed by 60 ml of a methanolic solution containing 10% of sodium acetate. The suspension obtained is left to sediment overnight, and is then filtered. The cake is dissolved in 20 ml of water and then re-precipitated with 60 ml of a methanolic solution containing 10% of sodium acetate. The suspension is filtered. The cake is dissolved again in 50 ml of water and the solution is then loaded into an MW 3500 cellulose ester membrane and dialyzed, changing the water baths regularly for 72 hours. The content of the membranes is lyophilized. 83 mg of a white lyophilized material are obtained. The yield obtained is 30%.

Proton spectrum in D₂O, 400 MHz, T=313 K, δ in ppm: 2.0 (3H, s), between 3.8 and 3.98 (4H, m), 4.03 (2H, m), 4.13 (1H, m), 4.18 (1H, m), 4.3 (1H, m), 4.6 (1H, broad s), 4.68 (1H, m), 4.8 (2H, m), 5.04 (1H, s).

### EXAMPLE 4

### Preparation of the Persulfated, Carboxyl-reduced Dermatan Sulfate Sodium Salt

(Compounds of formula (I) with the groups R₂=R₄=SO₃Na and R₃=H) 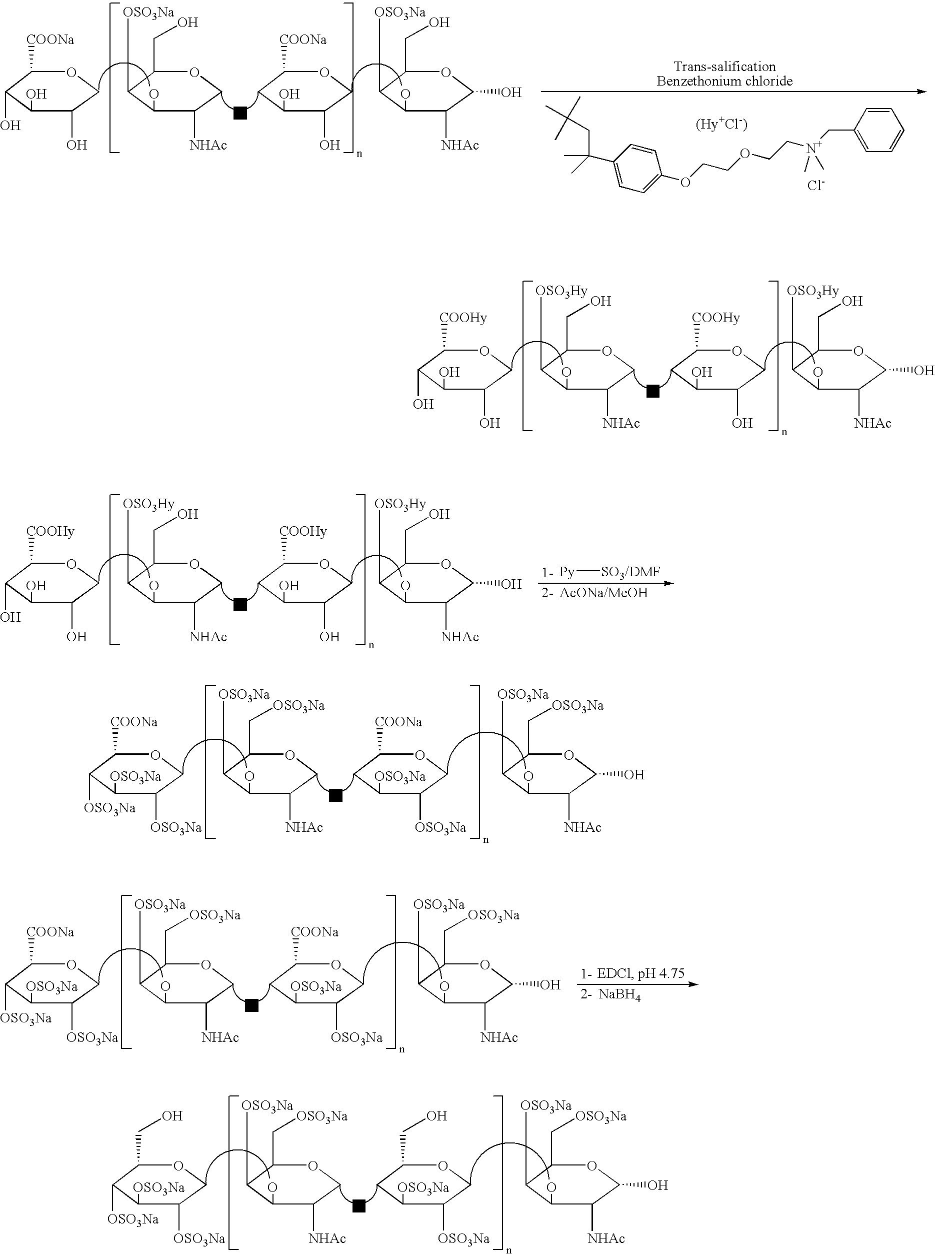
a) Preparation of the Dermatan Sulfate Benzethonium Salt

A solution of 11.6 g of benzethonium chloride in 85 ml of water is added to a solution of 5 g of dermatan sulfate sodium salt in 75 ml of water. The white suspension is filtered. The cake is washed with 6 portions of 150 ml of water, and then dried at 60° C. under reduced pressure (6 kPa) for approximately 72 h. 12 g of a cream solid are obtained. The yield obtained is 94%.

b) Preparation of the Persulfated Dermatan Sulfate Sodium Salt

4 g of dermatan sulfate benzethonium salt are dissolved in 80 ml of anhydrous dimethylformamide at a temperature in the region of 60° C. and under an inert atmosphere. After dissolution, a solution of 22.35 g of pyridine-sulfuric anhydride complex in 200 ml of anhydrous dimethylformamide is run in over approximately 45 minutes. After stirring for 1 hour 30 min, the reaction medium is cooled to a temperature in the region of 15° C., and then 50 ml of water are added. Once the addition is complete, 990 ml of a methanolic solution containing 10% of sodium acetate are added. The suspension is filtered. The solid obtained is dissolved in 200 ml of water and then re-precipitated with 600 ml of a methanolic solution containing 10% of sodium acetate. The suspension is filtered and the white solid obtained is then dissolved in 200 ml of water. The solution obtained is loaded into an MW 1000 cellulose ester membrane and dialyzed, changing the water baths regularly for 48 hours. The content of the membranes is lyophilized. 1.31 g of a white lyophilized material are obtained. The yield obtained is 51.8%.

Proton spectrum in D₂O, 400 MHz, T=303 K, δ in ppm: 2.02 (3H, s), 3.98 (3H, m), 4.21 (3H, m), 4.3 (1H, m), 4.67 (1H, m), 4.81 (1H, s), 4.9 (2H, m), 5.15 (1H, s).

c) Preparation of the Persulfated, Carboxyl-reduced Dermatan Sulfate Sodium Salt

A solution of 1.22 g of persulfated dermatan sodium salt in 190 ml of water is prepared at a temperature in the region of 20° C. The pH is adjusted to pH 4.7±0.1 with a 0.1N hydrochloric acid solution. 2.72 g of 1-ethyl-3-(3-dimethylaminopropyl)carbodiimide hydrochloride are added and the mixture is then stirred while maintaining the pH at 4.7±0.1. When the pH no longer changes, 11.1 g of sodium borohydride are then added in small portions. The reaction medium is stirred for approximately 20 hours at a temperature in the region of 20° C. After cooling to a temperature in the region of 4° C., the reaction medium is neutralized to pH 7 with concentrated hydrochloric acid (10N). The white suspension is loaded into an MW 1000 cellulose ester membrane and dialyzed, changing the water baths regularly for 20 hours. 1.2 1 of a methanolic solution containing 10% of sodium acetate are added to the content of the membranes (400 ml). The suspension is filtered and the cake is then washed with 3 portions of 100 ml of methanol. The white solid obtained is dissolved in water. The solution obtained is loaded into an MW 1000 cellulose ester membrane and dialyzed, changing the water baths regularly for 72 hours. The content of the membranes is lyophilized. 0.464 g of a white lyophilized material is obtained. The yield obtained is 39%.

Proton spectrum in D₂O, 400 MHz, T=333 K, δ in ppm: 2.0 (3H, s), 3.59 (2H, d, J=4 Hz), 3.8 (1H, s), between 3.85 and 4.02 (3H, m), 4.18 (2H, m), 4.27 (1H, m), 4.55 (1H, s), 4.65 (1H, broad s), 4.8 (1H, s), 4.88 (1H, s), 5.05 (1H, s).

### EXAMPLE 5

### Preparation of the 6-O-sulfated, Carboxyl-reduced Dermatan Sulfate Sodium Salt

(Compounds of formula (I) with the groups R₂=SO₃Na and R₃=R₄=H) 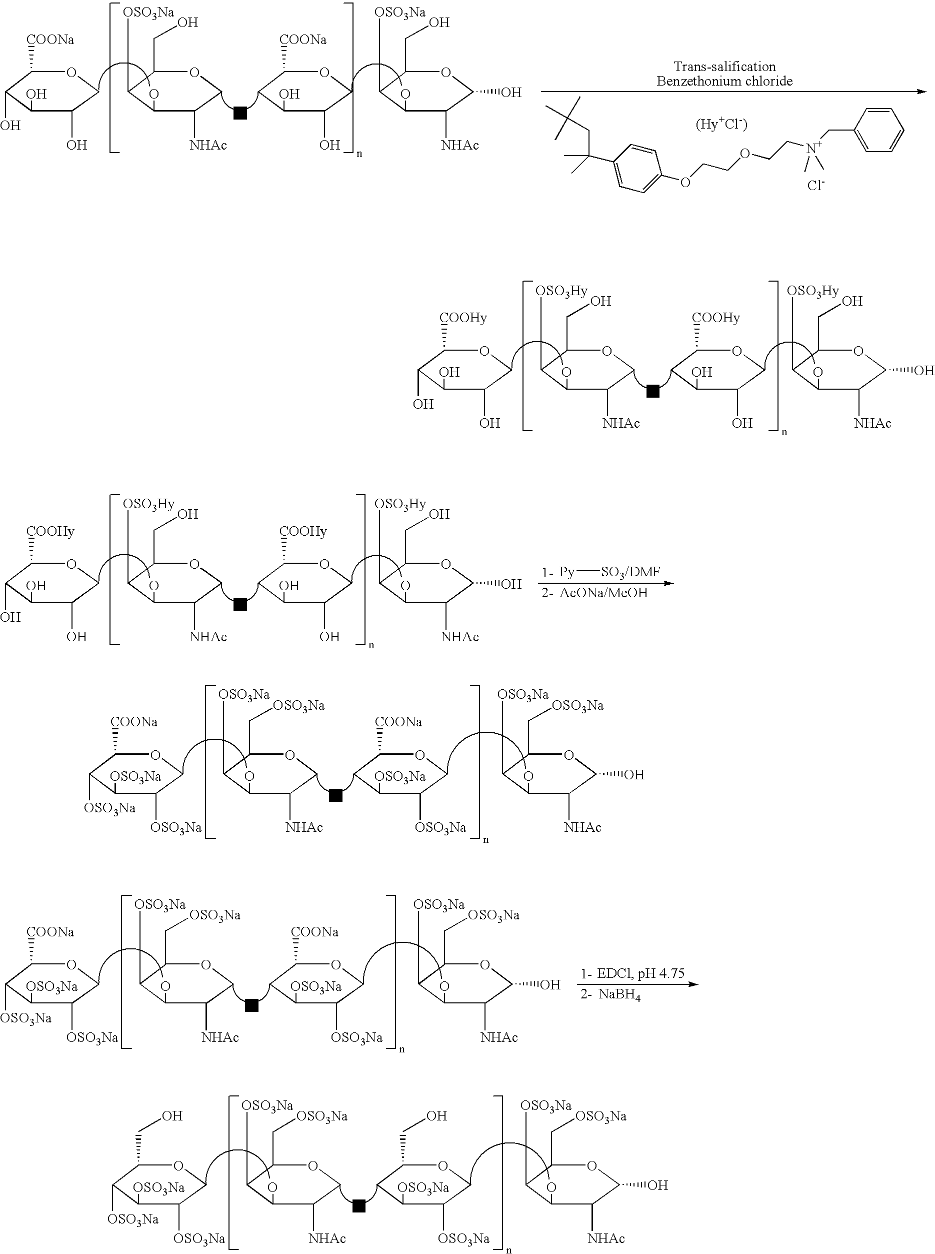
a) Preparation of the Dermatan Sulfate Benzethonium Salt

The dermatan sulfate benzethonium salt used in this example is common to that of example 4 a).

b) Preparation of the 6-O-sulfated Dermatan Sulfate Sodium Salt

4 g of dermatan sulfate benzethonium salt are dissolved in 80 ml of anhydrous dimethylformamide at a temperature in the region of 20° C. and under an inert atmosphere. After dissolution, the solution is cooled to a temperature in the region of 0° C. and a solution of 4.4 g of pyridine-sulfuric anhydride complex in 40 ml of anhydrous dimethylformamide is added. After stirring for 2 hours 30 minutes at a temperature in the region of 0° C., 60 ml of water are added to the reaction medium. Once the addition is complete, 600 ml of a methanolic solution containing 10% of sodium acetate are added. The suspension is filtered and the cake is washed with 30 ml of methanol. The solid obtained is dissolved in 200 ml of water and is then precipitated with 600 ml of a methanolic solution containing 10% of sodium acetate. After sedimentation for approximately 16 h, the mixture is filtered and the cake is washed with 20 ml of methanol. The white solid obtained is dissolved in 150 ml of water. The solution obtained is loaded into an MW 1000 cellulose ester membrane and dialyzed, changing the water baths regularly for 24 hours. The dialyzed solution is then lyophilized. 0.9 g of a white lyophilized material is obtained. The yield obtained is 47%.

Proton spectrum in D₂O, 400 MHz, T=328 K, δ in ppm: 2.0 (3H, s), 3.48 (1H, m), between 3.85 and 4.1 (5H, m), 4.18 (2H, m), 4.65 (3H, m), 4.8 (1H, s).

c) Preparation of the 6-O-sulfated, Carboxyl-reduced Dermatan Sulfate Sodium Salt

A solution of 0.8 g of 6-O-sulfated dermatan sulfate sodium salt in 75 ml of water is prepared at a temperature in the region of 20° C. The pH is adjusted to pH 4.7±0.1 with a 0.1N hydrochloric acid solution. 2.4 g of 1-ethyl-3-(3-dimethylaminopropyl)carbodiimide hydrochloride are added and the mixture is then stirred while maintaining the pH at 4.7±0.1.

When the pH no longer changes, 9.7 g of sodium borohydride are added in small portions. The reaction medium is stirred for approximately 20 hours at a temperature in the region of 20° C. After cooling to a temperature in the region of 15° C., the reaction medium is neutralized to pH 7 with concentrated hydrochloric acid (10N). The reaction mixture is loaded into an MW 1000 cellulose ester membrane and dialyzed, the water baths being changed regularly for 24 hours. 900 ml of a methanolic solution containing 10% of sodium acetate are added to the content of the membranes (300 ml). The suspension is filtered and the cake is dissolved in 50 ml of water. The solution thus prepared is loaded into an MW 1000 cellulose ester membrane and dialyzed, changing the water baths regularly for 50 hours. The content of the membranes is then lyophilized. 0.54 g of white lyophilized material is obtained. The yield obtained is 72%.

Proton spectrum in D₂O, 400 MHz, T=328 K, δ in ppm: 2.0 (3H, s), 3.5 (1H, m), 3.55 (3H, s), 3.7 (1H, s), 3.76 (1H, s), between 3.87 and 4.05 (3H, m), between 4.07 and 4.21 (2H, m), 4.3 (1H, m), 4.55 (1H, broad s), 4.72 (2H, m).

### EXAMPLE 6

### Preparation of the Carboxyl-reduced and Persulfated Dermatan Sulfate Sodium Salt

(Compounds of formula (I) with the groups R₂=R₃=R₄=SO₃Na)

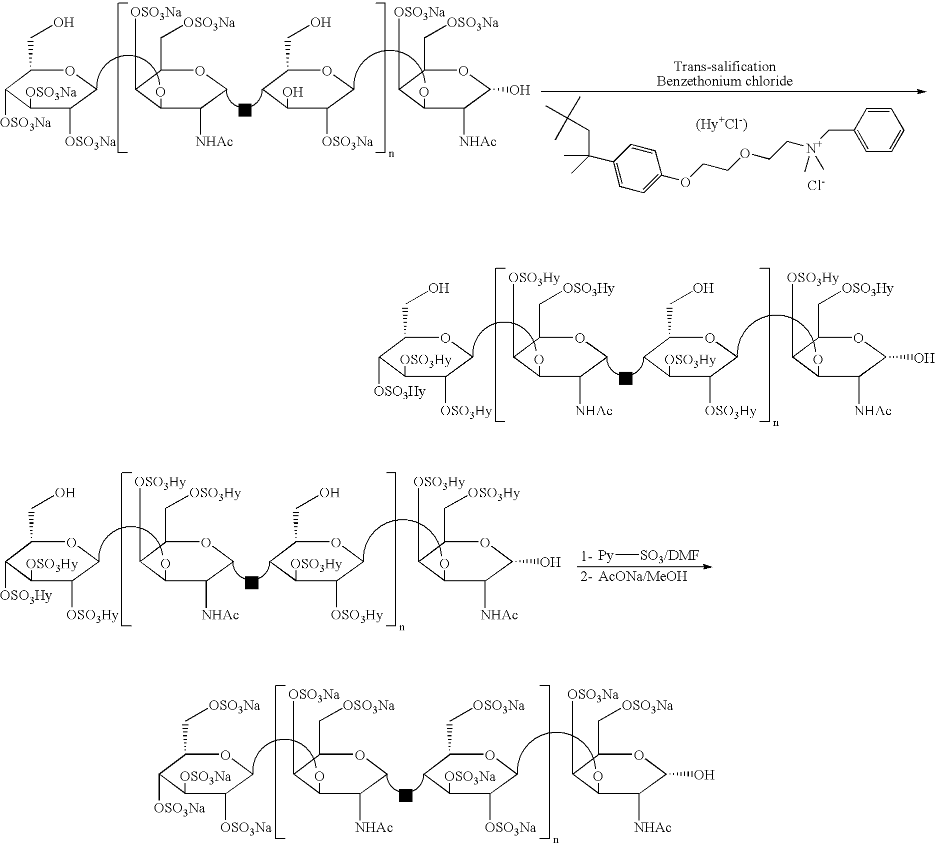

The dermatan derivative illustrating this example can be prepared from the persulfated, carboxyl-reduced dermatan sulfate sodium salt derived from example 4.

a) Preparation of the Persulfated, Carboxyl-reduced Dermatan Sulfate Benzethonium Salt

A solution of 0.65 g of benzethonium chloride in 10 ml of water is added to a solution of 0.28 g of persulfated, carboxyl-reduced dermatan sulfate sodium salt in 10 ml of water. The white suspension is filtered. The cake is washed with 5 portions of 40 ml of water, and then dried at 60° C. under reduced pressure (6 kpa) for approximately 48 hours. 0.685 g of a cream solid is obtained. The yield obtained is 81%.

b) Preparation of the Carboxyl-reduced and Persulfated Dermatan Sulfate Sodium Salt

0.685 g of persulfated, carboxyl-reduced dermatan sulfate benzethonium salt are dissolved in 8 ml of anhydrous dimethylformamide at a temperature in the region of 20° C. and under an inert atmosphere. After dissolution, a solution of 0.936 g of pyridine-sulfuric anhydride complex in 4 ml of anhydrous dimethylformamide is added. After stirring for 2 hours 30 min at a temperature in the region of 20° C., the reaction medium is cooled to a temperature in the region of 10° C. and then 6 ml of water are added. Once the addition is complete, 120 ml of a methanolic solution containing 10% of sodium acetate are added. The suspension is filtered and the cake is washed with 5 ml of methanol. The solid obtained is dissolved in 40 ml of water and is then precipitated with 120 ml of methanolic solution containing 10% of sodium acetate. The suspension is filtered and the white solid obtained is dissolved in 20 ml of water. The solution obtained is filtered through a 0.2 gm membrane and then loaded into an MW 1000 cellulose ester membrane and dialyzed, changing the water baths regularly for 24 hours. The content of the membranes is lyophilized. 0.128 g of a white lyophilized material is obtained. The yield obtained is 50%.

Proton spectrum in D₂O, 400 MHz, T=313 K, δ in ppm: 2.0 (3H, s), between 3.8 and 3.98 (4H, m), 4.03 (2H, m), 4.13 (1H, m), 4.18 (1H, m), 4.3 (1H, m), 4.6 (1H, broad s), 4.68 (1H, m), 4.8 (2H, m), 5.04 (1H, s).

### EXAMPLE 7

### Preparation of the Carboxyl-reduced, 6,6′-O-sulfated Dermatan Sulfate Sodium Salt

(Compounds of formula (I) with the groups R₂=R₃=SO₃Na and R₄=H)

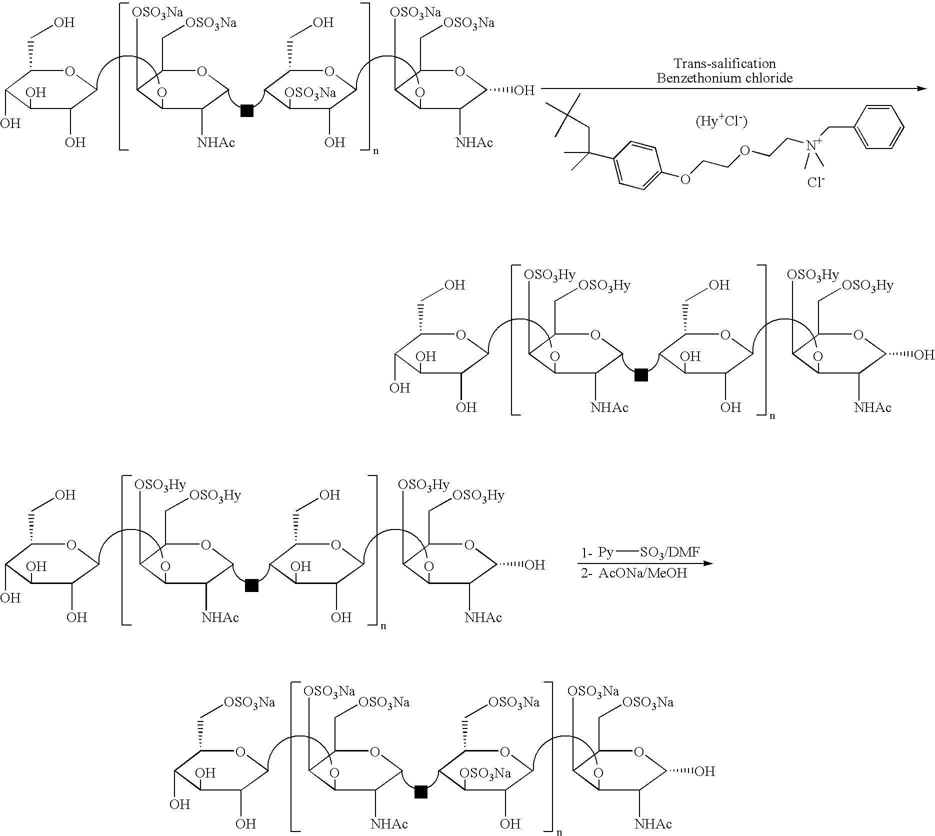

The carboxyl-reduced, 6-O-sulfated dermatan sulfate sodium salt used for this preparation is derived from example 5.

a) Preparation of the Carboxyl-reduced, 6-O-sulfated Dermatan Sulfate Benzethonium Salt

0.552 g of benzethonium chloride in 10 ml of water is added to a solution of 0.35 g of carboxyl-reduced, 6-O-sulfated dermatan sulfate sodium salt in 10 ml of water. The white suspension is filtered. The cake is washed with 6 portions of 10 ml of water and is then dried at ambient temperature under reduced pressure (6 kPa) and over potassium hydroxide for approximately 48 hours. 0.7 g of cream solid is obtained. The yield obtained is 84%.

b) Preparation of the Carboxyl-reduced, 6,6′-O-sulfated Dermatan Sulfate Sodium Salt

0.7 g of 6-O-sulfated, carboxyl-reduced dermatan sulfate benzethonium salt is dissolved in 11 ml of anhydrous dimethylformamide at a temperature in the region of 20° C. and under an inert atmosphere. After dissolution, a solution of 0.745 g of pyridine-sulfuric anhydride complex in 5.5 ml of anhydrous dimethylformamide is added, at a temperature in the region of −10° C. The reaction medium is stirred for 2 hours 30 min at a temperature in the region of −10° C. 7 ml of water are added. Once the addition is complete, 90 ml of a methanolic solution containing 10% of sodium acetate are added. The suspension is filtered and the cake is washed with 10 ml of methanol. The solid obtained is dissolved in 30 ml of water and is then precipitated with 90 ml of a methanolic solution containing 10% of sodium acetate. The suspension is filtered and the white solid obtained is washed with 5 ml of methanol and then dissolved in 10 ml of water. The solution obtained is loaded into an MW 3500 cellulose ester membrane and dialyzed, changing the water baths regularly for 24 hours. The content of the membranes is lyophilized. 0.252 g of a white lyophilized material is obtained. The yield obtained is 72%.

Proton spectrum in D₂O, 400 MHz, T=323 K, δ in ppm: 2.0 (3H, s), 3.49 (1H, m), 3.8 (1H, m), between 3.9 and 4.25 (8H, m), 4.45 (1H, m), 4.57 (1H, broad s), 4.75 (1H, s), 4, 4.80 (1H, s).

Pharmacological Test

Effect of the carboxyl-reduced derivatives according to the invention on the aggrecanase in synovial fluids or on preparations of recombinant ADAMTS protein.

The analysis is carried out on 96-well plates. Before the analysis, serial dilutions of the assay compounds are prepared in an aqueous solution.

Digestion:

In each well, a fixed volume of synovial fluid or aggrecanase activity generating a known increase, between 1.0 and 1.4 absorbance units (405 nm), under the conditions of the analysis, is mixed with 3 μl of solution of compound from the respective dilution step. Dulbecco's modified Eagle's medium (DMEM) is added to each well to give a final volume of 300 μl. The plate is then incubated for 1 hour at 37° C. in a carbon dioxide atmosphere.

After the addition of 5 μl of a solution of 1 μg/μl of recombinant substrate Agglmut in DMEM to each well (substrate as described by Bartnik E. et al., EP 785274, 1997), the reagent mixture is incubated for 4 hours at 37° C. under a carbon dioxide atmosphere.

Preparation of the analytical plate:

In a first step, the microplate is coated with 100 μl per well of a commercial goat anti-mouse IgG antibody for 1 hour at ambient temperature (5 μg/ml in a phosphate buffered saline solution, pH 7.4 [PBS buffer]). After washing with the PBS buffer containing 0.1% of Tween-20 (washing buffer), the wells are blocked by means of a step consisting of a one hour incubation with 100 μl per well of 5% bovine serum albumin in a PBS buffer containing 0.05% of Tween-20. Following another rinse with the washing buffer, each well is incubated with 100 μl of a solution, diluted to 1:1000, of BC-3 antibody in a PBS buffer containing 0.05% of Tween 20 and 0.5% of bovine serum albumin, at ambient temperature for one hour. This antibody recognizes the typical aggrecanase cleavage fragments (C. E. Hughes et al., Biochem J. 305 (3), 799-804, 1995).

Test procedure:

After another rinse of the analytical plate with the washing buffer, the complete set of mixtures originating from the preceding digestion is transferred to this plate, well by well, and incubated at ambient temperature for one hour. The plate is again washed as above. Next, 100 μl of the second antibody (anti-human IgG antibody labeled with peroxidase, 1:1000 in PBS containing 0.5% of BSA and 0.05% of Tween-20) are added to each well, with subsequent incubation at ambient temperature again for one hour. After a final rinse with the washing buffer, the color development is initiated by adding 100 μl of ABTS substrate solution (2 mg/ml, 2,2′-azinobis (3-ethylbenzothiazolinesulfonic acid) in 40 mM of sodium citrate plus 60 mM of disodium hydrogen phosphate, adjusted to a pH of 4.4 by adding acetic acid; 0.25 μl of 35% hydrogen peroxide added per ml immediately before measurement). The measurement is carried out by means of the screen mode using detection at 405 nm relative to a reference filter (620 nm) with automatic readings at 5-second intervals. The development is stopped as soon as the maximum signal (405 nm) in the absorbance range of 1.0 to 1.4 is reached.

TABLE 1

| Conc. of | % Conversion | | | |
|---|---|---|---|---|
| glycosaminoglycan | Example | Example | Example | Example |
| (μg/ml) | 3 or 6 | 2 or 7 | 4 | 5 |
| 1.0 | | | | 36.1 |
| 0.1 | 20.2 | 43.9 | 18.0 | 70.2 |
| 0.01 | 19.2 | 65.8 | 27.4 | 84.3 |
| 0.001 | 59.0 | 87.7 | 93.4 | 96.4 |
| 0.0001 | 96.7 | 96.5 | 100.0 | 100.0 |
| Estimated IC₅₀ | 0.0016 | 0.003 | 0.007 | 0.26 |
| (μg/ml): | | | | |

The estimated IC₅₀ for the product of example 1 (derivative that is carboxyl-reduced only) is of the order of 7.9 μg/ml

Effect of the Persulfated, Carboxyl-reduced Derivative (Example 3) on the Aggrecanase of Bovine Chondrocytes Cultured in Alginate Beads

In order to generate aggrecanase activity, the bovine chondrocytes cultured in gels of alginate matrix are stimulated with 10 ng/ml of IL-1α for 3 days in accordance with the method of C. E. Hughes et al., J. Biol. Chem. 273, 30576-30582, 1998.

In each well of a 96-well cell culture plate, 200 μl of chondrocyte supernatant containing aggrecanase activity are mixed with 100 μl of DMEM cell culture medium. At the concentrations to be tested, 5 μl of an aqueous solution of the product of example 2 are added in order to inhibit the aggrecanase activity, one hour before the addition of 5 μg of recombinant substrate Agglmut (E. Bartnik et al., EP 785274, 1997). The mixture is incubated at 37° C. for 17 hours and is then transferred into an ELISA plate in order to detect the neoepitopes generated by the aggrecanase activity, by binding of the BC-3 antibody as described previously (C. E. Hughes et al., Biochem. J. 305 (3), 799-804, 1995).

TABLE 2
(% inhibition of the conversion of the substrate by
aggrecanase activity in the supernatant)

| Concentration | |
|---|---|
| of the product | |
| of example 3 | % |
| (μg/ml) | Inhibition |
| 100 | 73.8 |
| 10 | 60.2 |
| 1 | 12.9 |
| 0.1 | 9.8 |
| 0.01 | 5.1 |

IC₅₀: 0.0059 mg/ml

Inhibition of Factor Xa

For the calibration, a standard sample of low molecular weight heparin (Enoxaparin) was used as reference.

The serial dilutions of the assay compound are prepared in a 0.046 M Tris buffer, pH 8.4, containing 0.15 M NaCl, 0.007 M EDTA, 0.1% of Tween 80 and 0.12 IU/ml of human antithrombin III. The samples of 50 μl of the respective dilutions are incubated with 50 μl of bovine factor Xa (13.6 U/ml) at 37° C. for 80 seconds. Next, 50 μl of chromogenic substrate, 1.1 mM S-2765, are added. The absorbance at 405 nm is measured in a photometer. The activity of the deblocked factor Xa is indicated by the emission of p-nitroaniline from the substrate.

When compared to the reference low-molecular weight heparin compound (100 U/mg), the glycosamonoglycans present a factor Xa, with much lower inhibitory activity:

| | Anti-factor Xa |
|---|---|
| Compound | activity |
| tested | (U/mg) |
| Examples 3, 6 | 2.6 |
| Examples 2, 7 | 0.6 |
| Example 4 | 1.1 |
| Example 5 | 0 |

Effect of the Persulfated, Carboxyl-reduced Derivative (Example 3) for Human Metalloproteases MNP-2 (Gelatinase-A) and MMP7

Commercial ELISA kits (such as those provided, for example, by the company Amersham Biosciences, kits RPN2617 and RPN2620) were used, respectively, to determine the inhibitory activity with respect to the MMP-2 and MMP-7 enzymes. The concentrations of the enzymes are, respectively, 800 ng/ml for MMP-2 and 300 ng/ml for MMP-7. The tests were carried out in accordance with the manufacturer's recommendations, by performing a series of dilutions of the compound to be studied in a PBS buffer, pH 7.5. The MMP-2 and MMP-7 enzymes were both inhibited in a concentration-dependent manner.

Derivative obtained according to example 3

| | |
|---|---|
| IC₅₀: | 0.6 μ/ml (effect on MMP-2) |
| | 0.04 μ/ml (effect on MMP-7) |

Effects of the Carboxyl-reduced Derivatives According to the Invention on Human Neutrophil Elastase

A commercially available enzyme (for example, human neutrophil elastase, Sigma No. E8140) is reconstituted in aliquots of 0.1 mg per vial with 0.276 ml of 50 mM sodium acetate buffer, pH 5.5, containing 200 mM NaCl (stock solution of the enzyme); 11 μl of this stock solution are diluted with 1.1 ml of the above HEPES buffer (enzyme assay solution).

The substrate solution is prepared by dissolving 118 mg of methoxy-succinyl-L-Ala-L-Ala-L-Pro-L-Val-p-nitro-anilide in 1 ml of DMSO (stock solution, for example Sigma No. M4765). In view of the application in the analysis, 9 μl of stock solution are then diluted in 1.19 ml of H₂O.

Serial dilutions of the assay compounds are prepared in 100 mM of HEPES buffer containing 500 mM of NaCl. The analysis is carried out on colorless 96-well polystyrene microplates (for example, Corning Costar, No. 3695.

10 μl of enzyme assay solution are mixed with 10 μl of the respective solution of diluted compound and 10 μl of the substrate solution. After incubation for 15 min, the cleavage of the substrate is read as increase in absorbance at 405 nm.

TABLE 4
Inhibitory activity on human neutrophil elastase

| Compound | IC₅₀ |
|---|---|
| tested | (μg/ml) |
| Examples 3, 6 | 0.08 |
| Examples 2, 7 | 0.12 |
| Example 4 | 0.11 |
| Example 5 | 10.0 |

1. A carboxyl-reduced and chemoselectively O-sulfated compound of dermatan sulfate, as a single compound or as a mixture thereof, or the salts thereof.

2. A carboxyl-reduced and sulfated compound of dermatan sulfate as claimed in claim 1, of formula (I): 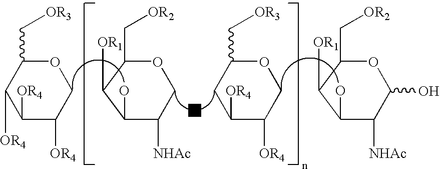
wherein R₁ is SO₃M, R₂, R₃ and R₄, which may be identical or different, are H or SO₃M, it being understood that at least one of the substituents R₂, R₃ and R₄ is a group SO₃M, n is an integer of between 0 and 150, M is an alkali metal, said compound isolated as a single compound or as a mixture, or the diastereoisomers thereof.

3. A carboxyl-reduced compound of dermatan sulfate as claimed in claim 2, wherein M is chosen from sodium, calcium, magnesium and potassium.

4. A carboxyl-reduced derivative of dermatan sulfate as claimed in claim 3, wherein M is sodium.

5. A carboxyl-reduced derivative of dermatan sulfate as claimed in claim 4, wherein R₂ and R₃ are SO₃Na and R₄ is H.

6. A carboxyl-reduced derivative of dermatan sulfate as claimed in claim 4, wherein R₂, R₃ and R₄ are SO₃Na.

7. A carboxyl-reduced derivative of dermatan sulfate as claimed in claim 4, wherein R₂ and R₄ are SO₃Na and R₃ is H.

8. A carboxyl-reduced derivative of dermatan sulfate as claimed 4, wherein R₂ is SO₃Na and R₃ and R₄ are H.

9. A process for preparing the carboxyl-reduced derivatives of dermatan sulfate as claimed in claim 1, comprising the following steps:
trans-salifying of dermatan sulfate with a quaternary ammonium salt,
sulfating the dermatan sulfate quarternary salt in organic medium, followed by salifying,
reducing the carboxyl of the persulfated dermatan sulfate
optionally a) by means of a carbodiimide derivative (so as to form an activated adduct) and in the presence of a reducing agent,
or b) by esterifying, the persulfated derivative being, where appropriate, trans-salified beforehand with a quaternary ammonium salt, reducing the corresponding ester derivative by the action of a reducing agent; and
where appropriate, trans-salifying the persulfated, carboxyl-reduced derivative with a quaternary ammonium salt, and then resulfating followed by salifying.

10. A process for preparing the carboxyl-reduced derivatives of dermatan sulfate as claimed in claim 1, comprising the following steps:
reducing the carboxyl of the dermatan sulfate sodium salt
optionally a) by means of a carbodiimide derivative (so as to form an activated adduct) and in the presence of a reducing agent,
or b) by esterifying, the dermatan sulfate sodium salt being, where appropriate, trans-salifying beforehand with a quaternary ammonium salt,
reducing the corresponding ester by the action of a reducing agent,
trans-salifying the carboxyl-reduced compound with a quaternary ammonium salt; and
sulfating in organic medium, followed by salifying.

11. The process as claimed in claim 9, wherein said organic medium is a complex of sulfuric anhydride with an organic base chosen from pyridine and trimethylamine.

12. The process as claimed in claim 11, wherein the dermatan sulfate quaternary ammonium salt to be sulfated is dermatan sulfate benzethonium salt, and the organic medium is about 10 to about 30 equivalents of pyridine-sulfuric anhydride complex per hydroxyl function to be sulfated, and at a temperature range of between about −15° C. and about 70° C.

13. The process as claimed in claim 10, wherein said organic medium is a complex of sulfuric anhydride with an organic base chosen from pyridine and trimethylamine.

14. The process as claimed in claim 13, wherein the dermatan sulfate quaternary ammonium salt to be sulfated is dermatan sulfate benzethonium salt, and the organic medium is about 10 to about 30 equivalents of pyridine-sulfuric anhydride complex per hydroxyl function to be sulfated, and at a temperature range of between about −15° C. and about 70° C.

15. The process as claimed in claim 10, further comprising sulfating trans-salified dermatan sulfate at a temperature of about 0° C. to obtain the 6-O-sulfated dermatan sulfate compounds or optionally at about 60° C. when it is desired to obtain the persulfated dermatan sulfate compounds.

16. The process as claimed in claim 10, further comprising sulfating carboxyl-reduced, trans-salified dermatan sulfate at a temperature of about 0° C. to obtain the 6,6′-O-sulfated compounds as defined in claim 5 wherein R₂ and R₃ are SO₃Na and R₄ is H or optionally at about 60° C. when it is desired to obtain the persulfated compounds as defined in claim 6 wherein R₂, R₃ and R₄ are SO₃Na.

17. The process as claimed in claim 9, further comprising (re)sulfating carboxyl-reduced, trans-salified, sulfated dermatan sulfate, compounds of formula (I) wherein R₂ and R₄ is SO₃Na and R₃ is H, as defined in claim 7 at a temperature of about 20° C. to obtain the persulfated compounds, of formula (I) wherein R₂, R₃ and R₄ are SO₃Na, as defined in claim 6.

18. The process as claimed in claim 9, further comprising (re)sulfating the carboxyl-reduced, trans-salified, 6-O-sulfated dermatan sulfate, compounds of formula (I) wherein R₂ is SO₃Na and R₃ and R₄ are H, at a temperature of about 10° C. to obtain the 6,6′-O-sulfated compounds, of formula (I) wherein R₂ and R₃ are SO₃Na and R₄ is H, as defined in claim 5.

19. The process as claimed in claim 9, wherein reducing the carboxyl is carried out in the presence of 1-(3-dimethylaminopropyl)-3-ethylcarbodiimide hydrocloride and an alkali metal borohydride.

20. The process as claimed in claim 19, wherein the amount of 1-(3-dimethylaminopropyl)-3-ethylcarbodiimide hydrochloride used, is within the range of from about 5 equivalents to about 20 equivalents per carboxyl and at a pH of between 4 and 5

21. The process as claimed in claim 20 wherein said pH range is between about 4.3 and about 4.9.

22. The process as claimed in claim 19 wherein said alkali metal borohydride is sodium borohydride.

23. The process as claimed in claim 10, wherein reducing the carboxyl is carried out in the presence of 1-(3-dimethylaminopropyl)-3-ethylcarbodiimide hydrochloride and an alkali metal borohydride.

24. The process as claimed in claim 23, wherein the amount of 1-(3-dimethylaminopropyl)-3-ethylcarbodiimide hydrochloride used, is within the range of from about 5 equivalents to about 20 equivalents per carboxyl and at a pH of between 4 and 5.

25. The process as claimed in claim 24 wherein said pH range is between about 4.3 and about 4.9.

26. The process as claimed in claim 23 wherein said alkali metal borohydride is sodium borohydride.

27. The process as claimed in claim 9, further comprising reducing the carbodiimide activated carboxyl adduct dermatan sulfate compound with about 10 to about 300 equivalents of alkali metal borohydride per equivalent of activated carboxyl of dermatin sulfate compound at a temperature range of between about 10° C. and about 70° C.

28. The process as claimed in claim 27 wherein said alkali metal hydride is sodium borohydride.

29. The process as claimed in claim 27 wherein the temperature range is between about 20° C. and about 50° C.

30. The process as claimed in claim 10, further comprising reducing the carbodiimide activated carboxyl adduct dermatan sulfate compound with about 10 to about 300 equivalents of alkali metal borohydride per equivalent of activated carboxyl of dermatin sulfate compound at a temperature range of between about 10° C. and about 70° C.

31. The process as claimed in claim 30 wherein said alkali metal hydride is sodium borohydride.

32. The process as claimed in claim 30 wherein the temperature range is between about 20° C. and about 50° C.

33. The process as claimed in claim 9, wherein in step b) the ester to be reduced, is methyl or benzyl, and said reducing agent is an alkali metal borohydride.

34. The process as claimed in claim 10, wherein in step b) the ester to be reduced, is methyl or benzyl, and said reducing agent is an alkali metal borohydride.

35. The process as claimed in claim 33, further comprising preparing said methyl ester from benzethonium salt of dermatan sulfate, in a solution of dichloromethane, with an amount in the range of about 2 to about 20 equivalents of methyl iodide per equivalent of benzethonium salt of dermatan sulfate.

36. The process as claimed in claim 35, wherein the amount of methyl iodide is in the range of about 4 to about 10 equivalents.

37. The process as claimed in claim 33, further comprising reducing the ester of dermatan sulfate with about 10 to about 300 equivalents of alkali metal borohydride at a temperature in the range of between about 10° C. and about 50° C.

38. The process as claimed in claim 37, further comprising reducing the ester with about 140 to about 250 equivalents of alkali metal borohydride at a temperature of about 20° C.

39. The process as claimed in claim 38 wherein said alkali metal borohydride is sodium borohydride.

40. The process as claimed in claim 34, further comprising preparing said methyl ester from benzethonium salt of dermatan sulfate, in a solution of dichloromethane, with an amount in the range of about 2 to about 20 equivalents of methyl iodide per equivalent of benzethonium salt of dermatan sulfate.

41. The process as claimed in claim 40, wherein the amount of methyl iodide is in the range of about 4 to about 10 equivalents.

42. The process as claimed in claim 34, further comprising reducing the ester of dermatan sulfate with about 10 to about 300 equivalents of alkali metal borohydride at a temperature in the range of between about 10° C. and about 50° C.

43. The process as claimed in claim 42, further comprising reducing the ester with about 140 to about 250 equivalents of alkali metal borohydride at a temperature of about 20° C.

44. The process as claimed in claim 43 wherein said alkali metal borohydride is sodium borohydride.

45. The process as claimed in claim 9, further comprising the following steps:
trans-salifying dermatan sulfate with benzethonium chloride,
sulfating the dermatan sulfate benzethonium salt in organic medium by means of a complex of sulfuric anhydride with an organic base chosen from pyridine and trimethylamine, followed by salifying with sodium acetate,
optionally reducing the carboxyl of dermatan sulfate sodium salt by reacting dermatan sulfate sodium salt with 1-(3-dimethylaminopropyl)-3-ethylcarbodiimide hydrochloride followed by reducing the resulting adduct with sodium borohydride,
or esterifying with methyl iodide dermatan sulfate compound trans-salified beforehand with benzethonium chloride,
reducing the methyl ester derivative with sodium borohydride
where appropriate, trans-salifying the persulfated, carboxyl-reduced derivative with benzethonium chloride; and
resulfating with the sulfuric anhydride-pyridine complex, followed by salifying with sodium acetate.

46. The process as claimed in claim 10, further comprising the following steps:
optionally reducing the carboxyl dermatan sulfate sodium salt by reacting dermatan sulfate sodium salt with 1-(3-dimethylaminopropyl)-3-ethylcarbodiimide hydrochloride followed by reducing the resulting adduct with sodium borohydride,
or esterifying by reacting methyl iodide with dermatan sulfate compound trans-salified beforehand with a quaternary ammonium salt,
reducing the methyl ester with sodium borohydride,
trans-salifying of the carboxyl-reduced derivative with benzethonium chloride; and
sulfating with the sulfuric anhydride-pyridine complex, followed by salifying with sodium acetate.

47. A process for obtaining a carboxyl-reduced and chemoselectively O-sulfated compounds of dermatan sulfate as claimed in claim 1, from the mixture of carboxyl-reduced compounds of dermatan sulfate further comprising fractionating by column chromatography on columns filled with polyacrylamide agarose gel or a polyacrylamide gel, said mixture being eluted with a sodium hydrogen carbonate solution, in a concentration of about 0.1 to about 1 mol/liter.

48. A pharmaceutical composition comprising a compound of claim 1 and one or more pharmaceutically acceptable excipients.

49. A method of treating a disease in a patient characterized by an increased activity in at least one of the matrix metalloproteinases selected from the group consisting of matrisylin (MMP-7), neutrophil elastase, aggrecanase hADAMTS1 and gelatinase A (MMP-2), comprising administering to said patient a therapeutically effective amount of a compound as claimed in claim 1.

50. The method of claim 49 wherein the disease is selected form the group consisting of joint degeneration, spondylosis, chondrolysis associated with joint trauma or prolonged immobilization of the joint, connective tissue disorders, wound healing conditions, periodontal disorders, chronic disorders of the locomotor system, arthropathies, myalgias and bone metabolism disorders.
